(19) United States
(12) Patent Application Publication
(10) Pub. No.: US 2012/0071972 A1
(43) Pub. Date: Mar. 22, 2012

(54) Multiphase eyecare

(76) Inventor: Iris Ginron Zhao, Normandy Park, WA (US)

(21) Appl. No.: 13/317,044

(22) Filed: Oct. 8, 2011

Related U.S. Application Data
(63) Continuation-in-part of application No. 12/657,023, filed on Jan. 12, 2010, now abandoned.

(30) Foreign Application Priority Data
Dec. 29, 2004 (CN) ZL20042000906.8
Feb. 16, 2005 (CN) ZL200420000977.5

(51) Int. Cl. A61F 2/16

(52) U.S. Cl. 623/6.13

(57) ABSTRACT

A novel multiphase optic lens contains an elastic chamber including a fluid or gel to transfer an eye positioning to a lens shape with a refractive power and visual axis matching an eye's targeting. The elasticity of an anterior wall of the elastic chamber is higher than that of the remainder of the wall of the elastic chamber, particularly in a near reading center at an inferior area toward the nose. A near response is achieved when the anterior wall is pushed anteriorly by the fluid or gel to form a protrusion when a user is looking downward and toward the nose, and vice versa for far response. A balance is achieved when the chamber is substantially perpendicular to horizon when a user is looking forward. This multiphase lens may be made in situ by laser. The method is useful for intraocular lens, nose/ear-free eyewear, and macular protection.

### CROSS-REFERENCE TO RELATED APPLICATION

This application is a CIP of co-pending U.S. patent application Ser. No. 12/657,023 filed Jan. 12, 2010.

### ACKNOWLEDGEMENT

Military has a right on it.

### BACKGROUND OF THE INVENTION

1. Field of the Invention

This invention relates broadly to a multiphase healthcare, particularly, a multiphase intraocular lens, which allows multi focusing through lens shape changing upon head and eye positioning.

2. State of the Art

Since Dr. Charlie Kelman, a Wills Eye Resident, invented the groundbreaking surgery of phacoemulsification in the mid-1960, it has become the most successful and common eye surgery. Numerous high quality mono and multifocal intraocular lenses (TOP) are used to replace human crystalline lens, e.g., AcryS of IQ Aspheric Natural IOL, Multifocal ReSTOR and Toric by Alcon Laboratories, Inc; Akreos™ Advanced Optics Aspheric lens by Bausch & Lomb; Tecnis Multifocal IOL by Abbott Medical Optics; Medical Design & Manufacturing West, Optimal Optic Eyewear™© by Multiphase Health, and Artisan® Phakic IOLs by OPHTEC.

So far, all lens including multifocal and accommodative lenses are in solid phase or a liquid medium controlled by a solid adjuster. Shall future eyecare and eye response in real life be limited by a solid? After decades of eye research, we are right at the edge where we can do so much more.

For example, currently, the two mainstreams for accommodation are:
a) An accommodative IOP capable of keeping posterior capsule-ciliary zonule-ciliary muscle function intact after implanting into the lens capsule;
b) An accommodative IOP capable of sliding over a carrier placed within an eye.

Now, a third new approach is created through multiphase co-action, which interchanges head and eye positioning to a lens configuration with a refractive power and axis according to the eye's targeting. The advantage is to integrative multi functions into one service and product, allowing accommodation power, visual axis adjusting, and light sensitivity similar to a young eye.

Dr. Phillips invented “Accommodating Intraocular Lens”, US 2003/0171808 disclosed an implanted lens adhered to the posterior capsule through a restraining element. It is a great improvement from prior accommodative lens which may damage the capsule and eye. However, even if 100% of the original accommodation is restored, aging does not stop. Most cataract patients lose accommodation not just due to the stiffness of the crystalline lens but also due to declined ciliary muscle and suspensory ligament. The natural accommodation power remained at age 64 and 68 is 1 D and 0.5 D respectively. The refractive power needed for 33 cm reading is 3 D. ⅓ of the power should be reserved for a comfortable reading (without headache or strain). To overcome aging, the elastic chamber of this multiphase lens includes a fluid or gel to obtain a high elasticity. Plus, such multiphase lens may be made in situ by an exterior laser.

Also, differed from US 2011/0040376, published Feb. 17, 2011, “Masked Intraocular Implanter and Lenses”, a solid lens invented by Christie et al, the present lens contains a fluid and gel. Comparing to a camera, the human iris is controlling the aperture of the pupil. Unless there is an iris defect, a mask before the iris can block normal iris function.

In view of Sanger's “Multifocal Ophthalmic Lens”, US Pub. No. 2010/0321632, where “ . . . power is altered discontinuously to have a stepwise power difference at a boundary between the far zone and the near zone; . . . ” the multiphase lens uses a liquid or gel to break the boundary between solid zones. This new lens offers an elastic configuration change to overcome the solid phase induced image waving in progressive lens and image jumping in multifocal lens.

Further differentiated from US Pub No. 2008/0097600 “Movable Ophthalmic Lens Assembly” taught by Hare, a lens moving over a surface of a carrier placed inside an eye, the present application anchor the lens in predesigned position.

Instead of combining multi sophisticated parts for one accommodative benefit, a reverse approach is used here, which allows a simple procedure and product for multi functions through a co-action among multi elements.

### SUMMARY OF THE INVENTION

It is therefore an object of the invention to introduce a multiphase eyecare through a simple multifunctional eyewear approach, especially a multiphase intraocular lens (IOP).

This multiphase IOP comprises an elastic chamber to induce near, far and middle response configurations with respective visual axis and refractive power changes according to the eye targeting. At the same time, another object of the invention is to reduce glare, wavefront abbreviation, and sudden strong light damage through using a dark edge and extended circular dark membrane on the peripheral margin of the lens. Plus, the extended membrane adheres to the capsule and reduces epithelium growth. It is also an object to eliminate unexpected implant movement.

Further object of the invention is to reduce imagine waving, jumping, ghosting, and depth confusion among plural focuses.

It is ultimate object of the invention to provide an IOP that allows routing implanting, easy positioning and trouble free follow-up. Moreover, this IOP can be formed in situ by a softener or liquidizer such as a laser light.

The lens is useful for multifunctional intraocular lens, exterior nose/ear free multiphase eyewear, contact lens, and macular protection. Alternatively, above lens may be inserted into a head mirror to protect a wearer's eye from incoming harm light.

### BRIEF DESCRIPTION OF THE DRAWINGS

FIG. 1 A, B, C show lateral views of the near, balanced, and far response configurations of the multiphase IOP. FIG. 1 D compares physiologic non-accommodation and accommodation responses from lateral views (Adler's Physiology of the Eye, Mosby).

FIG. 2A is a front view of prior symmetric IOP with a central optic axis. FIG. 2B is a front view of this asymmetric IOP with a descending optic center and extended dark edge. FIG. 2 C is a lateral view of eye-descended optical system (Bausch+Lomb, FDA clinical trial data).

FIG. 3A is a transverse view of prior symmetric IOP that focuses on a fovea. FIG. 3B is a transverse view of this asymmetric IOP that is deviating an incoming light from the fovea to a healthy area.

FIG. 4 A, B, C shows how a negative multiphase lens (for myopia) works. FIG. D is an exterior multiphase lens supported by a head band and positioned by a position marker.

FIG. 5 compares the head mirror of Otolaryngology and the multifunctional eyewear having a multiphase lens inserted into the central hole of a head mirror.

FIG. 6 shows how prior head mirror reflects incoming lights and how this new headwear protects a wearer's eye.

### DETAILED DESCRIPTION OF THE PREFERRED EMBODIMENTS

### Definition

Multiphase lens: The ability of the lens to adjust its shape based on the movement of the user's head and/or eye resulting in one of several lens configurations.

Multi-focusing: The ability of a multiphase eyeware device to focus incoming light on a user's retina based on a refractive visual axis and focusing power associated with a given configuration.

Multiphase co-action: The movement of the lens between different configurations.

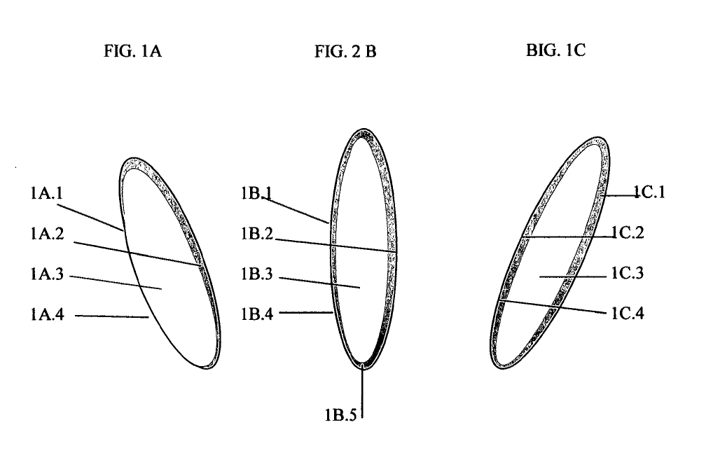

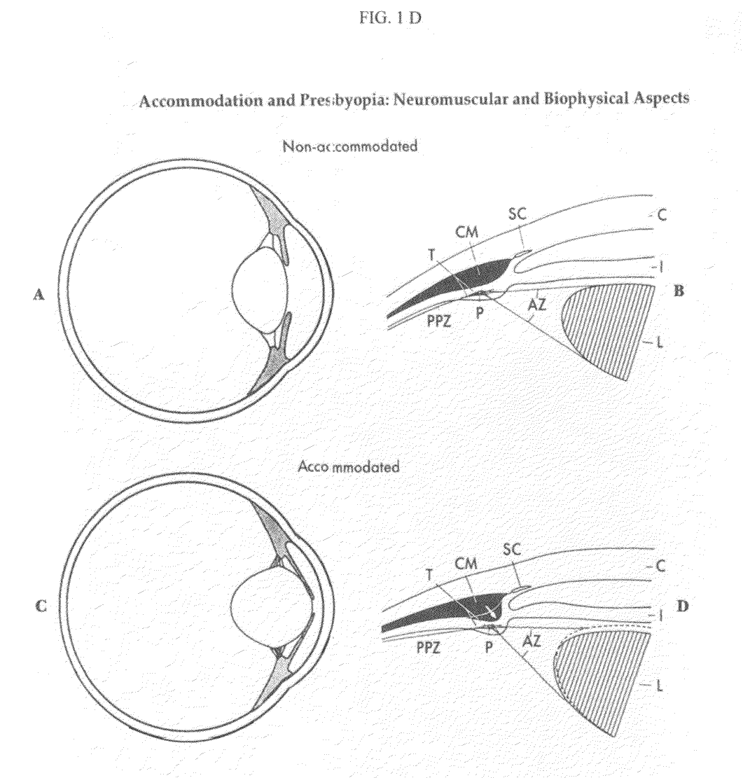

Turning now to FIG. 1, a first preferred embodiment according to the invention is a multiphase intraocular lens (IOP). The IOP has an optic axis and positive refractive power with an elastic chamber including a fluid or gel means for multiple focusing. (The fluid or gel can be filled into a predesigned elastic optic bag, or the elastic chamber can be formed during conditional lens mold through temperature or chemical control, or by coating a contour of a fluid when the fluid is shaped by a mold and frozen to a solid.)

The elasticity of the anterior wall of the elastic chamber is higher than the posterior wall of the elastic chamber, particularly in near reading center at inferior area toward the nose (inferior nasal quarter). For example, a thinner anterior wall (FIG. 1 A1,4; B1,4; C1,4) to protrude, or a higher permeability under pressure to allow volume expanding, or sensitive to certain light to expand. This lens is implanted into the remained capsule sac of a crystalline lens.

Referring to FIG. 1A, a near response configuration is achieved when the anterior wall (1A.1, 4) is pushed anteriorly by the interior fluid or gel (1A.3) to form a protrusion (1A.4) while a user is looking downward and toward the nose. In contrast to human lens capsule: anterior capsule thickness ≈8.2 μm and posterior capsule thickness ≈6.5 μm published by Ziebarth et al., the anterior wall (1A1,4; B1,4; C1,4) of this lens is thinner than posterior wall (1A.2, B2, C2) and thus, the accommodation is on the anterior wall. Since accommodating is only one element of near response complex, it shall not be singled out as a scope to bar other elements to work together. Once an IOP goes beyond solid phase, its elasticity can easily cover main accommodation power needs (+4 D).

Referring to FIG. 1 B, a middle response configuration is achieved when the anterior wall (1B.1, 4) is substantially perpendicular to horizon (balanced during standing) with an eccentric gravity close to the vertical line at 6 o'clock (1B.5) of the eye (toward the earth) while the eye is looking forward. 1B.2 is the posterior wall. 1B.3 is the fluid.

Referring to FIG. 1 C, a far response configuration is achieved when the anterior wall (1C.1, 4) is pulled back by the fluid or gel (1C.3) and the anterior surface (1C. 1, 4) is flatten while the user is looking upward and toward the tempora (upper temporal quarter). The posterior wall is thicker and stiffer (1C.2).

Now, a user's voluntary and involuntary eye positioning is interchanged to a lens configuration with a respective visual axis and refract power according to the eye's targeting to focus a near, middle, far light on a user's retina through a co-act.

In contrast to prior accommodating IOP, e.g., Crystalens made by Bausch & Lomb, which is controlled by ciliary muscles, the applicant adds gravity, elasticity and head-eye co-action to solve previous separated problems. For example, a near response includes not only accommodation power through ciliary muscle but also convergence (inward motion), miosis (pupil contract), and even further with eye rotation and head tilt in real life. Also, the method according to the invention allows eyecare in whole with coexisting problems, e.g., estropia in child, hyperopia induced severe headache, as a visual therapy.

### Reading Center (2B.8)

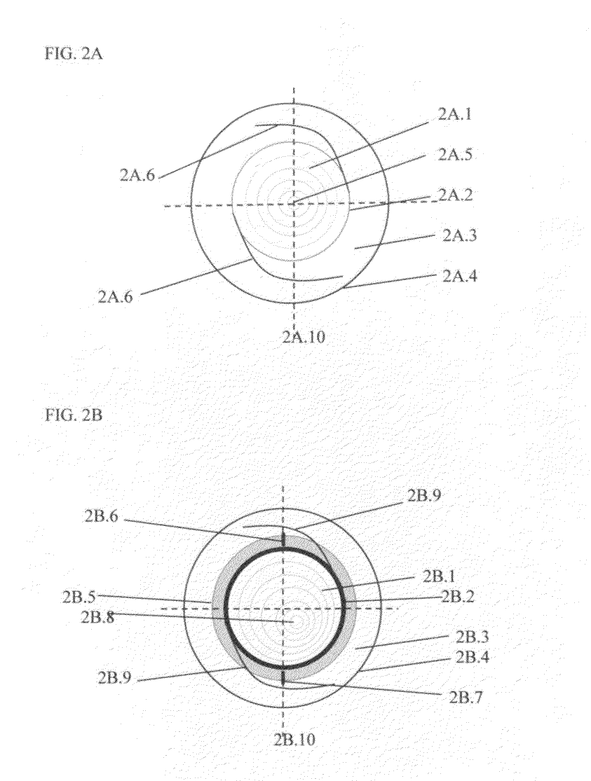

Referred to FIG. 2C and “Pupil decentration and iris tilting detected by Orbscan”, J Cataract Refrac Surg. 2005, this asymmetric IOP comprises a reading center having highest refractive power on its anterior surface at least 0.2 mm nasal decentration (2B.8) (eccentric from the geometric center of the lens). For far distance, normal eyes look upward and tempora, and thus, the lens is standing up. For people who read on bed, a reversed center may be more appreciated. Since the new IOP has eccentric gravity center, the lens is no longer free to move inside the eye. Because even a small dislocated refractive power and axis can cause headaches and asthenopia of accommodation.

Normal near response includes accommodation (The ciliary muscle contracts to allow the lens rounder and the pole protruding), accommodative convergence (synchronous symmetrical adduction of both eyes to focus on one object for binocular vision), miosis, and nasal decentration of pupils. The first sign of presbyopia may only be far sight blurred on the best eye after extended TV watching, reading or computer work. In view of eyecare, every detail counts. For example, a small word of “nasal” makes an asymmetric structure closer to normal reading complex.

Since nasal inferior descending for near reading did not draw attention from the crowd of IOP, it means difference. In view of instinct near vision complex, this multifunctional lens intends to integrate lens refractive power and optic axis with head and eye motion as young.

Next reference is made to FIG. 2, which compares the prior lens and the present lens in view of posterior capsules. The prior lens has a transparent edge (2A.2), an optic center at the geographic center (2A.5), and two haptics (2A.6). So, an edge glare may occur and a wide ring—shape transparent vacant is hollow out (2A.3) between the lens edge (2A.2) and the capsule edge (2A.4). 2A. 10 and 2B. 10 are vertical lines. To eliminate such glare and hollow, the multiphase lens (FIG. 2B) has a dark edge (2B.2) with an extended thin membrane-like skirt (2B.5). The transparent vacant and hollow (2B.3) between the lens edge (2B.2) and the capsule edge (2B.4) is reduced. This asymmetric lens contains a position marker (FIG. 2B.6≈12 o'clock and FIG. 2B.7≈6 o'clock) and an eccentric gravity center toward 6 o'clock (FIG. 2B.10) of a user's eye to reduce cycloposition, irritation, and rotation inside the eye. The two haptics are 2A.9s and 2B.9s respectively.

Average crystalline lens is about 3.5-4 mm thickness and 9 mm in diameter. The diameter of standard IOP ranges about 5.5-7 mm. Thus, there is a ring shaped window (hollow space, empty space) left after the natural lens is replaced by IOPs. The diameter of the pupil can be as small as 1.5-2 mm under the sun, 3-5 mm inside a room, and 6-8 mm in dark. So, a strong light can suddenly reach the retina through the hollow window during night driving. To reduce strong light damage, the dark edge of the new intraocular lens is extended to a 2 mm dark smooth elastic circular membrane that can be folded together with a lens during insertion and thereafter, is expanded and released to form an annular rim-shaped membrane. Another benefit of extended edge means for good adhesion with remained capsules and retarding epithelium growth after cataract surgery.

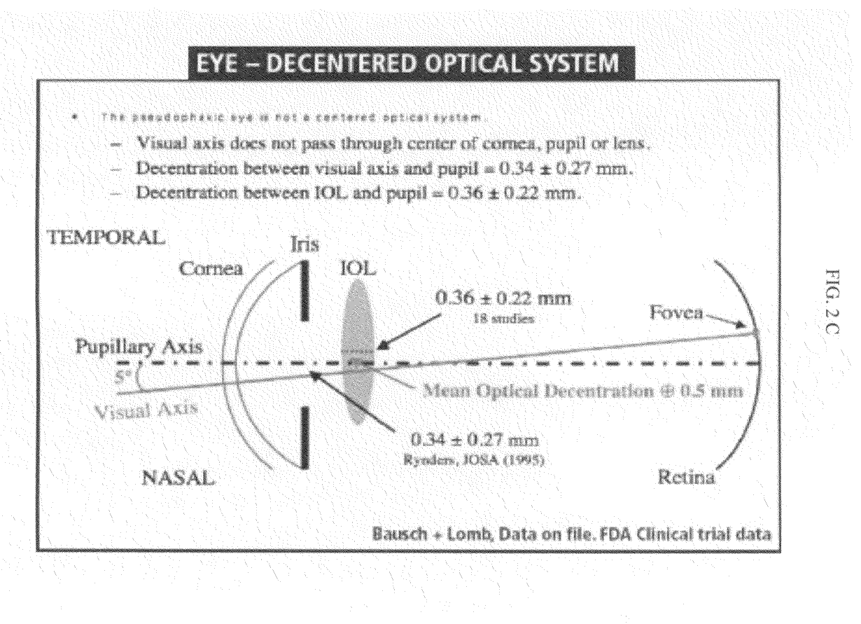

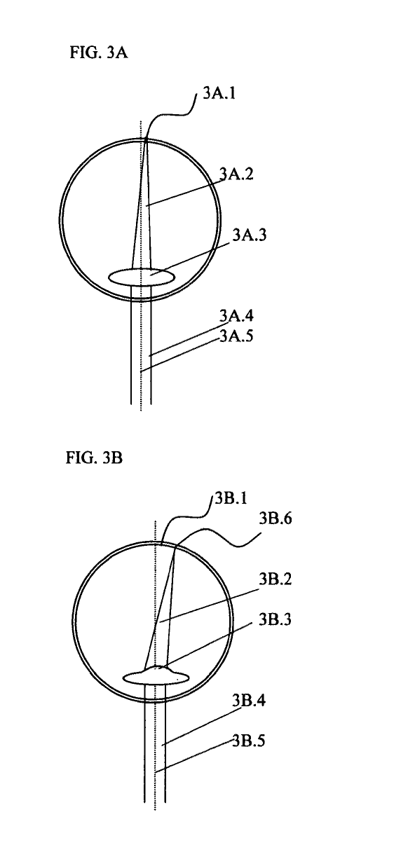

Reference is now made to FIG. 3, which is an IOP for reducing light damage on a penetrated retina hole at the fovea after watching the sun. FIG. 3A.3 is a prior IOP. 3A.2 is previous visual axis that is focusing incoming light (3A.4) on the fovea (3A.1). FIG. 3 B.3 is the new eccentric IOP that is deviating incoming light (3B.4) 10 degrees away from the fovea (3B1) to a healthy area (3B.6). The posterior protrusion (3B.3) creates an eccentric visual axis (3B.2). 3 A.5 and 3B.5 are the central line. This method is useful to a retina defect, e.g., macular hole, infarction, degeneration.

### Composition

Although many materials are available for an elastic IOP, e.g., silicone, acrylic, sodium hyaluronate, hydrogel plastics, polyphenyl ether liquid, polydimethylsiloxane, temperature gradient refractive index liquid, non-toxic Cargille optical liquid, pressure sensitive high index liquids, phosphate-saline, and phosphorus-sulfur-methylene liquid (C. D. West, Harvard University) etc, eye acceptance is the top priority.

For example, the fluid or gel may comprise a stem cell or embryonic cell.

### Yellow Colored Lens

Differed from red light that causes a surface feeling, blue color creates a continuous deep fancy feeling, attracting people to stair at it. Sharpest blue sense is at the fovea of macular area where is full of cones and looks slightly yellow. So, dark blue, violet and ultraviolet lights may harm the retina even before people can realize. In another side, bright blue light is critical for night sleep and helpful for reducing winter depression. Therefore, bright yellow (570-585 nm) is used to filter dark blue and re-emitted them to longer wave, e.g., bright blue and bluegreen to reduce macular degeneration.

Another goal is to filter dark red and re-emit them to brighter red, useful in dim light. However, once a yellow IOL is inserted into the eye, the color can not change according to the season or situation. So, it is safer to test a color and polarizing material on an exterior-ocular lens (EOL). Third goal of yellow lens is to narrow down wave front aberration and sharp imagine. Because a white light contains broad wavelengths. Each wavelength has its refractive index.

This method further polarizes incoming light, e.g., infrared light, to a visible range. The polarizer material is incorporated into the peri-central area so that infrared light can be seen through an enlarged pupil in dark

To remain natural lens and surrounding integrity, this application intends to keep the capsule intact. During applicant's cataract observation under electronic microscope, many opaque spots show debris and swollen crystalline fibers in a gel-like liquid. The debris reflects incoming light rather than allows incoming light to pass and therefore, an opacity is formed. Some dark hard nuclear cataract show condensed fibers and may contains pigments. The debris in gel or pigment in hard nucleus of opaque spots may be liquidized or softened to a fluid or gel in situ, e.g., by a laser light or a biocompatible chemical.

The top of the chamber may contain an air bubble. Both air bubble and gravity help the lens keep a balanced position.

The lens may contain a photochemical, ring-shaped zone, which is a gradient of a photochemical ranging from darkest at the edge of the elastic chamber to lightest at the center of the elastic chamber. The photochemical material is incorporated within the walls of the elastic chamber. Ideally, the aperture is shaped to a tilted vertical needle as a cat.

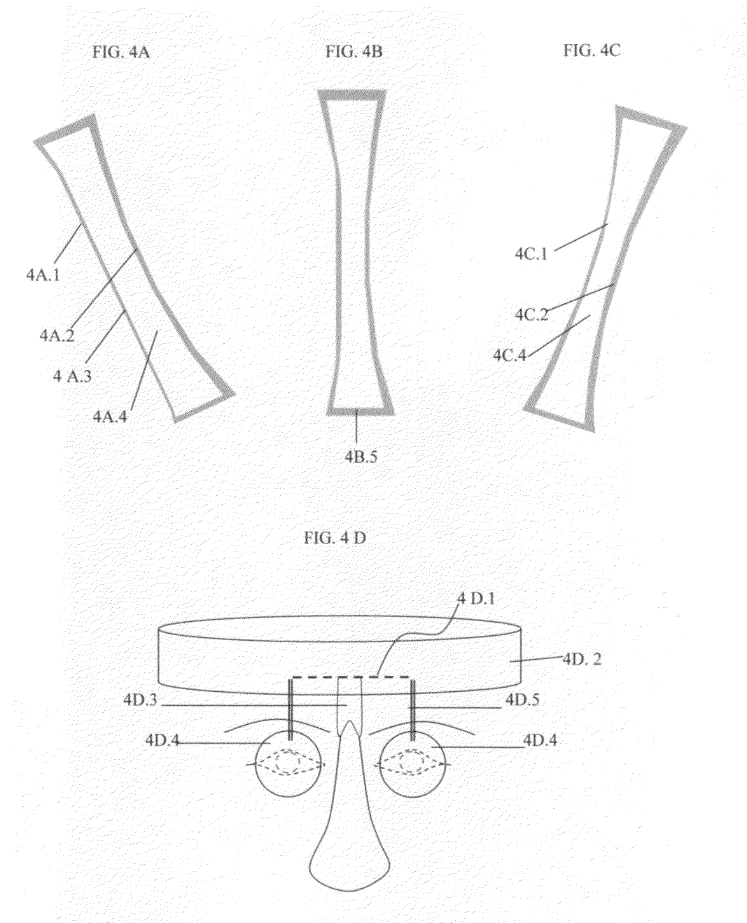

Referring now to FIG. 4, an embodiment of negative (concave) lens contains a fluid or gel. The anterior wall of the lens chamber is thinner than its posterior wall. FIG. 4A is a near response configuration. 4A.1 is the anterior wall. 4A.2 is the posterior wall. 4A.3 is the reading center pushed by interior gel (4A.4) to protrude. FIG. 4B is a middle position configuration. 4B.5 marks 6 o'clock position. FIG. 4C is a far response configuration. 4C.1 shows the thin anterior wall that is pulling back by the gel (4A.4) and 4C.2 is the thick stiff posterior wall. FIG. 4 D is an exterior multiphase eyewear connected to the head. Thus, most sensitive points in the nose roots and ear are released. 4D.1 fixes the pupil distance (PD). 4D.2 is a head support with positioning markers, e.g., foldable nose marker (4D.3), a central pupil line marker (4D.5). 4D.4 is a pair of exterior multiphase lenses.

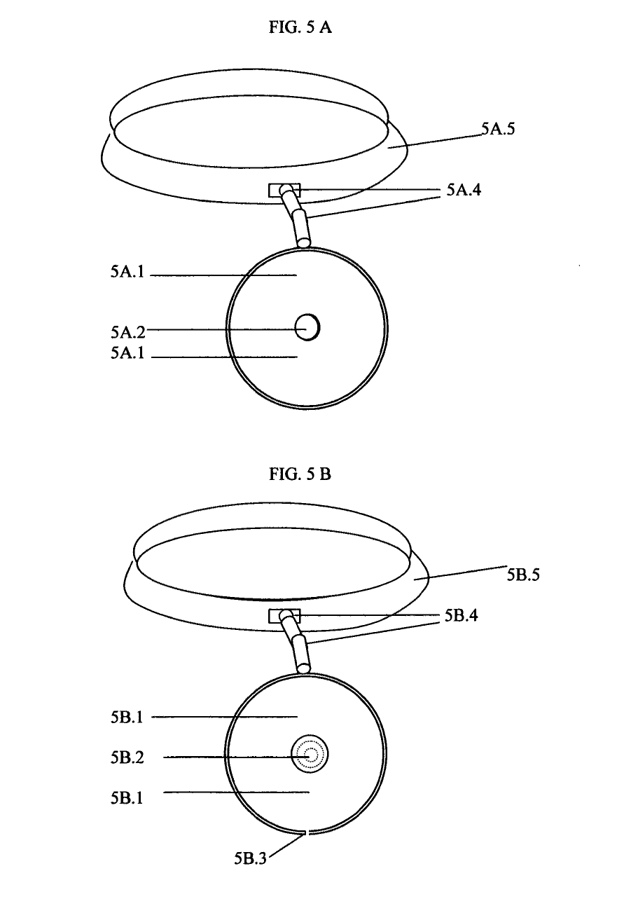

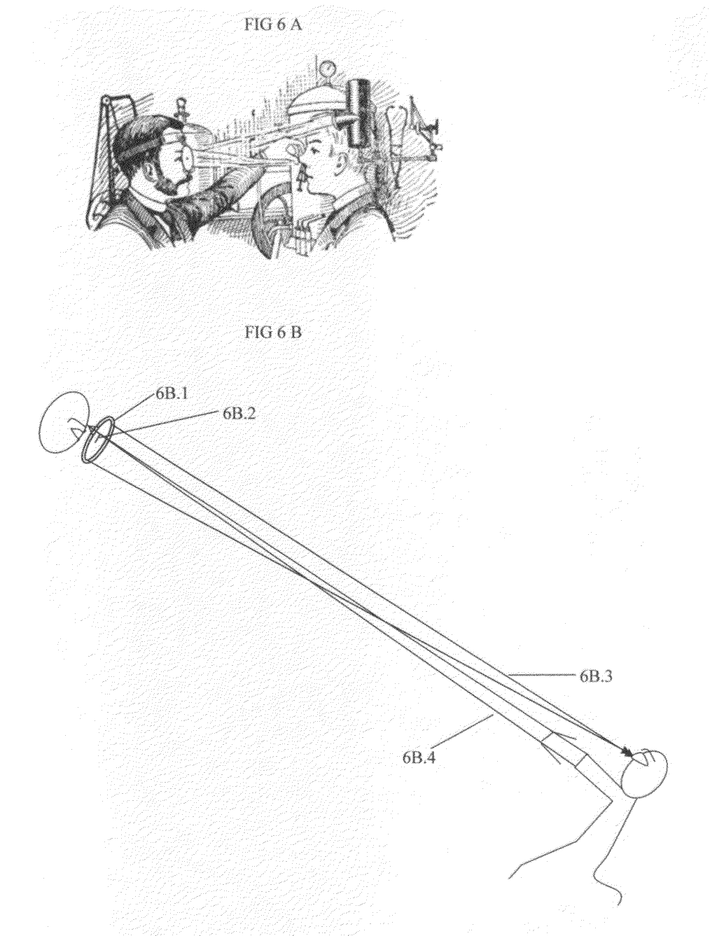

Turning now to FIG. 5A, a traditional head mirror (5A.1) contains an observing hole (5A.2) at its center (Am J Otolaryngology, 1982, 3:67-72). The mirror (5A.1) is used to illuminate a patient's deep passage (FIG. 6A, Wikipedia). 5A.4 and 5B.4 are joint connection to a head band (5A. 5; 5B. 5). Now, the mirror can be used in eyecare. The central hole is replaced by a multiphase yellow lens (5B.2) to reduce light damage. The surrounding mirror reflects incoming harm light back to the sender while helps the wearer to see through the multiphase lens inserted into the holes. The new eyewear is fixed to the head or upper body. 5B.1 is a concave annular mirror. 5B.3 is a positioning marker at 6 o'clock. 5B.4 is a joint connection. 5B.5 is a head band.

FIG. 6B shows how the mirror reflects incoming light back to the sender, e.g., laser, high beam light. 6 B.1 is the new defense eyewear. 6B.2 is the central protective lens. 6B.3 is reflective light. 6B.4 is the incoming light.

Since almost half of any population needs some degree of vision correction in life, multiphase lens should offer more benefits beyond natural aging.

It is the goal of present art to integrate unused evidences (element, act, operation) to a co-act through multiphase interchange. For example, once the head is out of the fix band of examination room or laboratory, both human and animals enjoy head/eye moving, which shall be allowed to join the new eyecare in this application.

Multiphase Interchange may change an opacity area of cataract to a transparent gel or bring multidisciplinary element into a simple service or product. Multiphase Eyecare™© allows transferring a stiff phase to a flexible phase.

It is appreciated that the present invention is not limited by what has been particularly shown and described herein. The scope of the present invention includes both combinations of the characters described herein as well as modifications and variations of embodiments, approaches and theories which are not permitted in the prior art.

What is claimed is:

1. A method of providing a multiphase eyecare using a multiphase eyewear comprising:
a) providing a lens comprising an elastic chamber containing a fluid or gel for multi focusing;
wherein the lens comprises an optic axis with a refractive power;
wherein the elasticity of an anterior wall of said elastic chamber is higher than that of the remainder of the wall of the elastic chamber, particularly in a near reading center at an inferior area toward the nose;
b) positioning said lens on a visual axis within the eye;
wherein a near response configuration is achieved when said anterior wall is pushed anteriorly by said fluid or gel to form a protrusion when a user is looking downward and toward the nose;
wherein a far response configuration is achieved when the anterior wall is pulled back by the fluid or gel means for flatting an anterior surface when said user is looking upward and toward the tempora;
wherein a middle response configuration is achieved when the lens is substantially perpendicular to the horizon when the user is looking forward;
wherein the user's voluntary and involuntary eye movements result in the lens being in either the near response configuration, the far response configuration, or the middle response configuration;
wherein the near, far, and middle response configurations each comprise a respective visual axis and refractive power to focus incoming light on a user's retina.

2. The method of claim 1 wherein said lens comprises a position marker and an eccentric gravity center toward 6 o'clock position of said user's eye.

3. The method of claim 1 wherein said lens comprises a yellow composition.

4. The method of claim 1 wherein said lens polarizes an incoming light to 465-780 nm wavelengths.

5. The method of claim 1 wherein said providing includes a step of liquidizing or softening an opaque area within a cataract.

6. The method of claim 1 further comprising positioning said lens into the remained capsule sac of an eye after a previous lens is removed following pupil dilation and ciliary muscle relaxing.

7. The method of claim 6 wherein said lens comprises a dark edge with an extension of a 0.5 mm-3 mm wide dark elastic membrane curving approximately 60°-360° around said dark edge in a ring-shape

8. The method of claim 6 wherein said lens comprises an eccentric visual axis that deviates incoming light at least 5 degrees away from the visual axis of a natural lens to a related healthy area.

9. The method of claim 6 wherein said fluid or gel comprises a stem cell or embryo cell.

10. A multiphase lens comprising:
a lens comprising an elastic chamber containing a fluid or gel for multi focusing;
wherein said lens comprises an optic axis with a refractive power;
wherein the elasticity of an anterior wall of said elastic chamber is higher than that of the remainder of the wall of the elastic chamber, particularly in a near reading center at an inferior area toward a user's nose;
wherein said lens is sized to be positioned within said user's eye;
wherein a near response configuration is achieved when the anterior wall is pushed anteriorly by said fluid or gel to form a protrusion when the user is looking downward and toward the nose;
wherein a far response configuration is achieved when the anterior wall is pulled back by the fluid or gel means for flatting an anterior surface when the user is looking upward and toward the tempora;
wherein a middle response configuration is achieved when the lens is substantially perpendicular to the horizon when the user's eye is looking forward;
wherein the user's voluntary and involuntary eye movements result in the lens being either the near response configuration, the far response configuration, or the middle response configuration;
wherein the near, far, and middle response configurations each comprise a respective visual axis and refractive power to focus incoming light on a user's retina.

11. The lens of claim 10 wherein the lens is asymmetric, comprising a position marker and an eccentric gravity center toward 6 o'clock position of said user's eye.

12. The lens of claim 10 further comprises a yellow composition.

13. The lens of claim 10 further comprising a material that polarizes incoming light to 465-780 nm wavelengths.

14. The lens of claim 10 is made in situ by a laser light, wherein said laser light is focusing on an opaque area within a cataract.

15. The lens of claim 10 wherein the lens is inserted into a remained capsule sac of the eye after a previous lens is removed.

16. The lens of claim 10 further comprising a dark edge with an extension about 0.5 mm-3 mm wide dark elastic membrane curving approximately 60°-360° around said dark edge.

17. The lens of claim 10 further comprising an eccentric visual axis that deviates incoming light at least 5 degrees away from previous visual axis to a related healthy area.

18. The lens of claim 10 wherein the top of said lens chamber includes an air bubble.

19. The lens of claim 10 wherein said fluid or gel comprises a stem cell or embryo cell.
